(19) United States
(12) Patent Application Publication
(10) Pub. No.: US 2004/0133177 A1
(43) Pub. Date: Jul. 8, 2004

(54) Barrier performance of absorbent article components

(76) Inventors: Jerry Zucker, Charleston, SC (US); Nick Mark Carter, Mooresville, NC (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/666,297

(22) Filed: Sep. 18, 2003

Related U.S. Application Data
(60) Provisional application No. 60/411,645, filed on Sep. 18, 2002.

(51) Int. Cl. A61F 13/15; A61F 13/20

(52) U.S. Cl. 604/370

(57) ABSTRACT

The present invention is directed to absorbent articles comprised of one or more components exhibiting an improved barrier to basis weight performance. The improved barrier performance of the aforementioned absorbent article components is a result of supplying a strong and durable nonwoven substrate layer followed by deposition of a nano-denier, essentially continuously filament barrier layer onto the nonwoven substrate layer thereby providing absorbent articles that exhibit enhanced barrier performance in comparison to conventional absorbent articles.

### TECHNICAL FIELD

The present invention relates generally to a disposable absorbent article with an improved barrier performance and more specifically, to disposable absorbent hygiene articles wherein one or more component of the hygiene article have improved barrier performance relative to basis weight due to the use of nonwoven fabrics comprised of continuous nano-fiber filaments.

### BACKGROUND OF THE INVENTION

Nonwoven fabrics are used in a wide variety of applications where the engineered qualities of the fabrics can be advantageously employed. Hygiene applications, and particularly disposable absorbent articles, such as sanitary napkins, diapers, and incontinence devices, utilize nonwovens as components, including, but not limited to, backsheets, topsheets, surge layers, core wraps, and leg cuffs. Certain components, backsheets and leg cuffs specifically, require barrier properties so as to prevent any undesirable leaking that may soil the user's clothing. A preferred nonwoven fabric for such components is that of a continuous filament laminate, such as a spunbond/meltblown/spunbond construct.

In and of themselves, continuous filament fabrics are relatively highly porous, and ordinarily require an additional component in order to achieve the required barrier performance. Typically, barrier performance, as measured by hydrostatic head, has been enhanced by the use of a barrier “meltblown” layer of micrometer scale filaments, which are drawn and fragmented by a high velocity air stream, and deposited into a self-annealing mass. Typically, such a meltblown layer exhibits very low porosity, enhancing the barrier properties of compound fabrics formed with spunbond and subsequent meltblown layers. Such nonwoven constructs have been utilized as barrier fabrics as disclosed in U.S. Pat. No. 4,041,203 to Brock et al., the disclosure of which is herein incorporated by reference.

Conventional spunbond/meltblown/spunbond (SMS)-type fabrics for barrier applications, as represented by the aforementioned disposable hygiene products, are manufactured in a basis weight range of 60-65 grams per square meter, typically relying upon a meltblown layer of more than 10 grams per square meter, to provide the desired barrier function. Ordinarily, these types of fabrics have a hydrostatic head rating of greater than 45 centimeters, before the addition of topical treatments.

Further prior art improvements on the SMS construct have been made by incorporating multiple lightweight meltblown barrier layers, i.e. SMMS fabrics, in lieu of a single heavyweight meltblown layer. Fabrication in this manner has been found to reduce hydrostatic head failures, which can otherwise result due to defects that are common in meltblown fabrics; the plural meltblown layers compensate for defects, which may exist in any one layer. While multiple meltblown layers act to facilitate manufacturing efficiency, the complexity of such a process requires additional equipment for each subsequent layer. Further, the final basis weight of the multiple meltblown layers remains at approximately the same level as practiced with a single, heavier weight meltblown layer.

U.S. Pat. No. 5,464,688 teaches the use of modified polypropylene resin with a higher melt flow rate to produce a meltblown web having average fiber diameters of from 1 to 3 microns and pore sizes distributed in the range from 7 to 12 microns compared to previously reported meltblown webs, which have pore sizes distributed predominantly in the range from 10 to 15 microns.

U.S. Pat. No. 5,482,765 teaches the addition of fluorocarbons to either the meltblown or spunbond layer and a meltblown layer with between 5 and 20% polybutylene. Such modifications provide a laminate having improved barrier and strength to weight ratios. The enhancement is measured by the ratio of hydrostatic head to meltblown layer basis weight of greater than 115 cm/osy (3.38 cm/gsm).

The provision of one or more nano-denier filament layers significantly improves the overall barrier performance of the compound fabric (which includes both laminate and composite constructs) utilized as absorbent article components while, optionally, reducing the weight of the overall construct, and can be utilized as an alternative to various performance enhancing coatings and costly or complicated treatments. The nano-denier spunbond layer also provides a more uniform interface between the layers during the manufacture of a compound nonwoven fabric resulting in further improved barrier performance in the fabricated article.

The present invention contemplates an absorbent article with improved barrier properties due to the incorporation of a nano-fiber nonwoven construct in one or more components of the absorbent article.

### SUMMARY OF THE INVENTION

The present invention is directed to absorbent articles comprised of one or more components exhibiting an improved barrier to basis weight performance. The improved barrier performance of the aforementioned absorbent article components is a result of supplying a strong and durable nonwoven substrate layer followed by deposition of a nano-denier, essentially continuously filament barrier layer onto the nonwoven substrate layer thereby providing absorbent articles that exhibit enhanced barrier performance in comparison to conventional absorbent articles.

A barrier layer preferentially comprising nano-fibers of infinite length, wherein the average fiber diameter of the nano-fiber is in the range of less than or equal to 1000 nanometers, and preferably less than or equal to 500 nanometers, is applied to at least one substrate layer. Said substrate layer or layers and said nano-fiber layer layers, and optionally one or more secondary barrier materials, are consolidated into a single compound fabric.

The thermoplastic polymers of the nano-denier continuous filament barrier are chosen from the group of thermoplastic polymers consisting of polyolefins, polyamides, and polyesters, wherein the polyolefins are chosen from the group consisting of polypropylene, polyethylene, and combinations thereof. It is within the purview of the present invention that the nano-denier, continuous filament barrier layer or layers may comprise either the same or different thermoplastic polymers. Further, the nano-denier continuous filaments of the barrier layer or layers may comprise homogeneous, bicomponent, and/or multi-component profiles, as well as, performance modifying additives, and the blends thereof.

The strong and durable carrier substrate layer comprises a material selected from suitable media, such media being represented by, but not limited to: continuous filament nonwoven fabrics, staple fiber nonwoven fabrics, continuous filament or staple fiber woven textiles, and films. The composition of the substrate layer may be selected from synthetic and natural materials and the blends thereof. In a fabric formed in accordance with the present invention, the incorporation of one or more nano-denier barrier layers provide substantial improvement in barrier function, allowing for reduction in the total amount of the substrate and/or barrier layer required to meet barrier performance criteria.

A further aspect of the present invention is directed to the nano-denier barrier layer providing a more uniform support layer for subsequently applied barrier layers or substrate layers during the manufacturing process, thus providing an improvement in barrier function of the resulting absorbent article components.

Formation of fabrics from nano-denier barrier materials, particularly when a light basis weight nano-denier barrier layer is either coated or “dusted” onto a substrate layer or is combined with one or more conventional barrier layers, can provide enhanced barrier properties. The present invention allows for the production of a same weight fabric with improved barrier properties or a lighter weight fabric that is suitable for use as a barrier fabric, particularly for disposable absorbent article applications, such as diapers and incontinence devices.

Other features and advantages of the present invention will become readily apparent from the following detailed description, the accompanying drawings, and the appended claims.

### BRIEF DESCRIPTION OF THE DRAWINGS

FIG. 1 is a top plan view, partially cut-away, of a disposable absorbent article, illustrated as a disposable diaper, embodying the present invention.

### DETAILED DESCRIPTION

While the present invention is susceptible of embodiment in various forms, there will hereinafter be described, presently preferred embodiments, with the understanding that the present disclosure is to be considered as an exemplification of the invention, and is not intended to limit the invention to the specific embodiments disclosed herein.

The present invention is directed to an absorbent article with an improved barrier performance due to the incorporation one or more components comprised of nano-denier continuous filaments and at least one substrate layer of strong and durable carrier substrate. In order to achieve desired barrier properties to weight ratios for the backsheet and/or leg cuff structure, the nano-denier continuous filaments preferably have a denier of less than or equal to 1000 nanometers, and preferably have a denier less than or equal to about 500 nanometers.

Suitable nano-denier continuous filament barrier layers can be formed by either direct spinning of nano-denier filaments or by formation of a multi-component filament that is divided into nano-denier filaments prior to deposition on a substrate layer. U.S. Pat. No. 5,678,379 and No. 6,114,017, both incorporated herein by reference, exemplify direct spinning processes practicable in support of the present invention. Multi-component filament spinning with integrated division into nano-denier filaments can be practiced in accordance with the teachings of U.S. Pat. No. 5,225,018 and No. 5,783,503, both incorporated herein by reference.

Technologies capable of forming a strong and durable carrier substrate layer include those which form continuous filament nonwoven fabrics, staple fiber nonwoven fabrics, continuous filament or staple fiber woven textiles (to include knits), and films. A substrate is determined to be strong and durable based upon the substrate having sufficient physical properties to withstand manufacturing and fabrication processes. Fibers and/or filaments comprising the strong and durable carrier substrate layer are selected from natural or synthetic composition, of homogeneous or mixed fiber length. Suitable natural fibers include, but are not limited to, cotton, wood pulp and viscose rayon. Synthetic fibers, which may be blended in whole or part, include thermoplastic and thermoset polymers. Thermoplastic polymers suitable for blending with thermoplastic resins include polyolefins, polyamides and polyesters. The thermoplastic polymers may be further selected from homopolymers; copolymers, conjugates and other derivatives including those thermoplastic polymers having incorporated melt additives or surface-active agents.

In general, continuous filament nonwoven fabric formation involves the practice of the spunbond process. A spunbond process involves supplying a molten polymer, which is then extruded under pressure through a large number of orifices in a plate known as a spinneret or die. The resulting continuous filaments are quenched and drawn by any of a number of methods, such as slot draw systems, attenuator guns, or Godet rolls. The continuous filaments are collected as a loose web upon a moving foraminous surface, such as a wire mesh conveyor belt. When more than one spinneret is used in line for the purpose of forming a multi-layered fabric, the subsequent webs are collected upon the uppermost surface of the previously formed web. The web is then at least temporarily consolidated, usually by means involving heat and pressure, such as by thermal point bonding. Using this means, the web or layers of webs are passed between two hot metal rolls, one of which has an embossed pattern to impart and achieve the desired degree of point bonding, usually on the order of 10 to 40 percent of the overall surface area being so bonded.

Staple fibers used to form nonwoven fabrics begin in a bundled form as a bale of compressed fibers. In order to decompress the fibers, and render the fibers suitable for integration into a nonwoven fabric, the bale is bulk-fed into a number of fiber openers, such as a garnet, then into a card. The card further frees the fibers by the use of co-rotational and counter-rotational wire combs, then depositing the fibers into a lofty batt. The lofty batt of staple fibers can then optionally be subjected to fiber reorientation, such as by air-randomization and/or cross-lapping, depending upon the ultimate tensile properties of the resulting nonwoven fabric desired. The fibrous batt is integrated into a nonwoven fabric by application of suitable bonding means, including, but not limited to, use of adhesive binders, thermobonding by calender or through-air oven, and hydroentanglement.

The production of conventional textile fabrics is known to be a complex, multi-step process. The production of staple fiber yarns involves the carding of the fibers to provide feedstock for a roving machine, which twists the bundled fibers into a roving yarn. Alternately, continuous filaments are formed into bundle known as a tow, the tow then serving as a component of the roving yarn. Spinning machines blend multiple roving yarns into yarns that are suitable for the weaving of cloth. A first subset of weaving yarns is transferred to a warp beam, which, in turn, contains the machine direction yarns, which will then feed into a loom. A second subset of weaving yarns supply the weft or fill yarns which are the cross direction threads in a sheet of cloth. Currently, commercial high-speed looms operate at a speed of 1000-1500 picks per minute, whereby each pick is a single yarn. The weaving process produces the final fabric at manufacturing speeds of 60 inches to 200 inches per minute.

The formation of finite thickness films from thermoplastic polymers, suitable as a strong and durable carrier substrate layer, is a well-known practice. Thermoplastic polymer films can be formed by either dispersion of a quantity of molten polymer into a mold having the dimensions of the desired end product, known as a cast film, or by continuously forcing the molten polymer through a die, known as an extruded film. Extruded thermoplastic polymer films can either be formed such that the film is cooled then wound as a completed material, or dispensed directly onto a secondary substrate material to form a composite material having performance of both the substrate and the film layers. Examples of suitable secondary substrate materials include other films, polymeric or metallic sheet stock, and woven or nonwoven fabrics.

Extruded films utilizing the composition of the present invention can be formed in accordance with the following representative direct extrusion film process. Blending and dosing storage comprising at least one hopper loader for thermoplastic polymer chip and, optionally, one for pelletized additive in thermoplastic carrier resin, feed into variable speed augers. The variable speed augers transfer predetermined amounts of polymer chip and additive pellet into a mixing hopper. The mixing hopper contains a mixing propeller to further the homogeneity of the mixture. Basic volumetric systems such as that described are a minimum requirement for accurately blending the additive into the thermoplastic polymer. The polymer chip and additive pellet blend feeds into a multi-zone extruder. Upon mixing and extrusion from the multi-zone extruder, the polymer compound is conveyed via heated polymer piping through a screen changer, wherein breaker plates having different screen meshes are employed to retain solid or semi-molten polymer chips and other macroscopic debris. The mixed polymer is then fed into a melt pump, and then to a combining block. The combining block allows for multiple film layers to be extruded, the film layers being of either the same composition or fed from different systems as described above. The combining block is connected to an extrusion die, which is positioned in an overhead orientation such that molten film extrusion is deposited at a nip between a nip roll and a cast roll.

When a secondary substrate material is to receive a film layer extrusion, a secondary substrate material source is provided in roll form to a tension-controlled unwinder. The secondary substrate material is unwound and moves over the nip roll. The molten film extrusion from the extrusion die is deposited onto the secondary substrate material at the nip point between the nip roll and the cast roll to form a strong and durable carrier substrate layer. The newly formed substrate layer is then removed from the cast roll by a stripper roll and wound onto a new roll.

It is within the purview of the present invention that a secondary barrier material can be combined with the nano-denier barrier layer. Suitable secondary barrier materials can be selected from such representative materials as: meltblown, microporous films and monolithic films.

A related means to the spunbond process for forming a layer of a nonwoven fabric is the meltblown process. Again, a molten polymer is extruded under pressure through orifices in a spinneret or die. High velocity air impinges upon and entrains the filaments as they exit the die. The energy of this step is such that the formed filaments are greatly reduced in diameter and are fractured so that microfibers of finite length are produced. This differs from the spunbond process whereby the continuity of the filaments is preserved. The process to form either a single layer or a multiple-layer fabric is continuous, that is, the process steps are uninterrupted from extrusion of the filaments to form the first layer until the bonded web is wound into a roll. Methods for producing these types of fabrics are described in U.S. Pat. No. 4,043,203. The meltblown process, as well as the cross-sectional profile of the spunbond filament or meltblown microfiber, is not a critical limitation to the practice of the present invention.

Breathable barrier films can be combined with the improved barrier performance imparted by combining the breathable barrier film with nano-denier continuous filaments. Monolithic films, as taught in U.S. Pat. No. 6,191,211, and microporous films, as taught in U.S. Pat. No. 6,264,864, both patents herein incorporated by reference, represent the mechanisms of forming such breathable barrier films.

It is believed that by providing a nano-denier continuous layer upon which a subsequent secondary barrier layer may deposited, several enhancements of the fabric can be realized. For a given basis weight of the spunbond layer, a finer denier fabric will give a greater number of filaments and a smaller average pore size per unit area. The smaller average pore size will result in a more uniform deposition of the secondary barrier material onto the nano-denier barrier layer. A more uniform secondary barrier layer will also have fewer weak points in the web at which a failure in barrier performance can occur. The nano-denier barrier layer also serves to support the secondary barrier layer structurally in the compound nonwoven material. A nano-denier barrier layer provides a smaller average pore size and a larger number of support points for the secondary barrier layer, this results in shorter spans of unsupported secondary barrier material. This mechanism embodies the well-known concept that reduction in the average span length results in enhanced structural integrity.

Manufacture of nonwoven compound fabrics embodying the principles of the present invention includes the use of fibers and/or filaments having different composition. Differing thermoplastic polymers can be compounded with the same or different performance improvement additives. Further, fibers and/or filaments may be blended with fibers and/or filaments that have not been modified by the compounding of additives.

Utilizing the above-discussed substrate and barrier layer manufacturing technologies, combinations of different constructs can be combined with a nano-denier barrier layer to yield compound nonwoven materials of further improved barrier performance.

Disposable waste-containment garments are generally described in U.S. Pat. No. 4,573,986, No. 5,843,056, and No. 6,198,018, which are incorporated herein by reference.

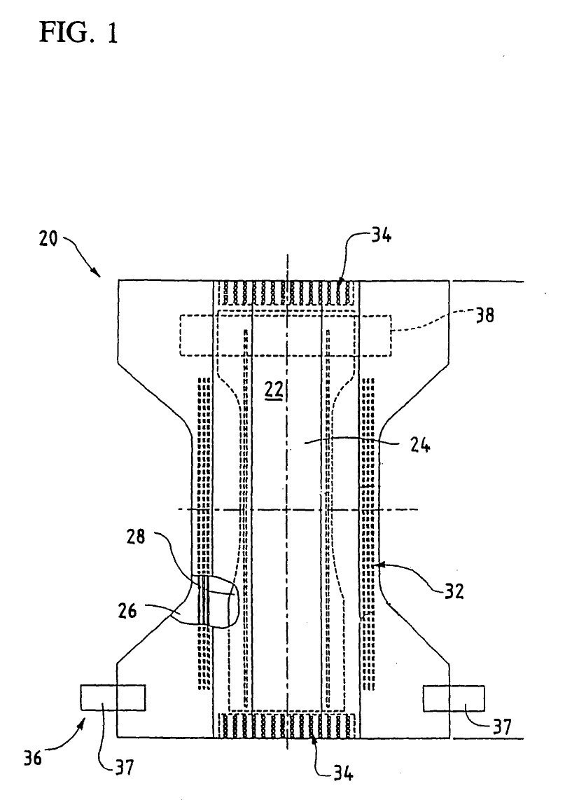

An absorbent article incorporating an improved barrier fabric of the present invention is represented by the unitary disposable absorbent article, diaper 20, shown in FIG. 1. As used herein, the term “diaper” refers to an absorbent article generally worn by infants and incontinent persons that is worn about the lower torso of the wearer. It should be understood, however, that the present invention is also applicable to other absorbent articles such as incontinence briefs, incontinence undergarments, diaper holders and liners, feminine hygiene garments, sanitary napkins, training pants, pull-on garments, and the like.

FIG. 1 is a plan view of a diaper 20 in an uncontracted state (i.e., with elastic induced contraction pulled out) with portions of the structure being cut-away to more clearly show the construction of the diaper 20. As shown in FIG. 1, the diaper 20 preferably comprises a containment assembly 22 comprising a liquid pervious topsheet 24; a liquid impervious backsheet 26 joined to the topsheet; and an absorbent core 28 positioned between the topsheet 24 and the backsheet 26. The absorbent core 28 has a pair of opposing longitudinal edges, an inner surface and an outer surface. The diaper can further comprise elastic leg features 32; elastic waist features 34; and a fastening system 36, which preferably comprises a pair of securement members 37 and a landing member 38.

It is within the purview of the invention that one or more components comprised of nano-denier continuous filaments may be incorporated into an absorbent article. A layer of the nano-denier continuous filaments may be utilized in various components of the same absorbent article, including, but not limited to, the backsheet, leg cuff, ears, tabs, and/or tummy panel of an absorbent article. Practical application of an improved barrier fabric comprising a nano-denier barrier layer as described in this invention for backsheet 26 and elastic leg feature 32, or leg cuff, results in a absorbent article that is lighter in weight while maintaining performance. A lighter weight backsheet material, in addition to a lighter weight leg cuff, is expected to be more flexible and therefore more conforming to deformation of the overall structure as the absorbent article is applied and worn.

When the end-use article is a feminine hygiene product, such as a sanitary napkin or the like, the improved barrier performance nano-denier layer may be incorporated into various components wherein a barrier performance is desired. Typically, a sanitary napkin utilizes an absorbent facing material. Further, the napkin contains an absorbent core, which may comprise absorbent fibrous material such as comminuted wood pulp fibers, cotton linters, rayon fibers, cotton staples, and the like. A layer of absorbent material typically envelops the core of the napkin. The napkin will also normally contain a fluid impervious layer on the side normally worn away from the body, wherein the fabric of the invention can be employed.

From the foregoing, numerous modifications and variations can be effected without departing from the true spirit and scope of the novel concept of the present invention. It is to be understood that no limitation with respect to the specific embodiments disclosed herein is intended or should be inferred. The disclosure is intended to cover, by the appended claims, all such modifications as fall within the scope of the claims.

What is claimed is:

1. A disposable absorbent article, comprising;
an absorbent core,
a liquid pervious topsheet,
a liquid impervious backsheet,
said liquid impervious backsheet comprising a nonwoven compound fabric, and
said nonwoven compound fabric comprises a nano-denier barrier layer comprising a plurality of continuous thermoplastic filaments having a denier of less than about 1000 nanometers, a secondary barrier layer, and a substrate layer.

2. A disposable absorbent article, comprising;
an absorbent core,
a liquid pervious topsheet,
a liquid impervious backsheet,
a liquid impervious leg cuff,
said liquid impervious backsheet comprising a nonwoven compound fabric, and
said nonwoven compound fabric comprises a nano-denier barrier layer comprising a plurality of continuous thermoplastic filaments having a denier of less than about 1000 nanometers, a secondary barrier layer, and a substrate layer.

3. A disposable absorbent article, comprising;
an absorbent core,
a liquid pervious topsheet,
a liquid impervious backsheet,
a liquid impervious leg cuff,
said liquid impervious leg-cuff comprising a nonwoven compound fabric, and
said nonwoven compound fabric comprises a nano-denier barrier layer comprising a plurality of continuous thermoplastic filaments having a denier of less than about 1000 nanometers, a secondary barrier layer, and a substrate layer.
